(19) United States
(12) Patent Application Publication
(10) Pub. No.: US 2025/0235557 A1
(43) Pub. Date: Jul. 24, 2025

(54) METHOD OF GENE TARGETING UTILIZING OUTER MEMBRANE VESICLE

(71) Applicant: The Research Institute at Nationwide Children's Hospital, Columbus, OH (US)

(72) Inventors: Lexie Blalock, Columbus, OH (US); Lauren Warren, Westerville, OH (US); Christian Lauber, Hilliard, OH (US)

(21) Appl. No.: 18/855,791

(22) PCT Filed: May 12, 2023
(86) PCT No.: PCT/US23/22021

§ 371 Date: Oct. 10, 2024

Related U.S. Application Data
(60) Provisional application No. 63/341,019, filed on May 12, 2022.

(51) Int. Cl. A61K 48/00; A61K 9/50; A61K 38/18; A61P 25/00; A61P 29/00; C12N 15/88

(52) U.S. Cl. CPC A61K 48/005; A61K 9/5068; A61K 38/18; A61P 25/00; A61P 29/00; C12N 15/88

(57) ABSTRACT

A method of gene targeting utilizing outer membrane vesicle is disclosed. As described herein outer membrane vesicles (OMVs) have the ability to modulate the expression of NRG1 intracellularly, which affects intracellular NRG1 mediated functions in addition to autocrine and paracrine signaling that support cell development, differentiation and growth. The OMVs are useful in modifying the expression of NRG1 and the expression of genes other than NRG1. The Pg OMVs also function as a gene therapy vector, as it is up taken by mammalian cells and crosses both the placental and blood brain barrier.

### CROSS-REFERENCE TO RELATED APPLICATIONS

The present application claims priority to U.S. Provisional Patent Application Ser. No. 63/341,019 filed May 12, 2022 entitled METHOD OF GENE TARGETING UTILIZING OUTER MEMBRANE VESICLE. The entire contents of the above-identified application are incorporated herein by reference in their entireties for all purposes.

### FIELD OF THIS DISCLOSURE

The present disclosure relates to a method of gene targeting utilizing outer membrane vesicle (OMV). More specifically, the present disclosure relates to utilizing bacterially derived OMVs to modulate expression of specific mammalian genes.

### BACKGROUND

Outer membrane vesicles (OMVs) are produced by Gram-negative bacteria that are blebbed outward from a cell and are encased in a lipid bilayer composed of an outer leaflet of lipopolysaccharides (LPS) and an inner membrane of phospholipids. OMVs encapsulate a range of bacterial components that include, proteins, nucleic acids, LPS, toxins, and secondary metabolites. OMVs are traffickable within a host and will attach to bacteria and host cells to deliver their cargo in a concentrated manner. One producer of OMVs is Porphyromonas gingivalis (Pg). However, the nucleic acid profiles of Pg OMVs have not been characterized.

Additionally, mammals have a Neuregulin-1 (NRG1) gene. NRG1 is a growth factor whose isoforms function as agonists of an ErbB receptor. NRG1 regulates brain development, myelination, neuronal migration, and survival. Dysregulation of NRG1 has been associated with blood brain barrier (BBB) mis-function, cancer, and a variety of behavior disorders, such as, schizophrenia, autism spectrum disorder, Alzheimer's Disease, and bipolar disorder.

### SUMMARY

One aspect of the present disclosure includes a method of altering NRG1 expression in a subject in need thereof, the method comprising isolating an outer membrane vesicle (OMV) from Porphyromonas gingivalis (Pg), the Pg OMV containing transfer ribonucleic acid (tRNA) complementary to NRG1 messenger ribonucleic acid (mRNA), and administering to the subject the Pg OMV.

Another aspect of the present disclosure includes a gene therapy vector for expressing an exogenous nucleic acid sequence comprising an outer membrane vesicle (OMV) from Porphyromonas gingivalis (Pg), a nucleic acid sequence encoding for a protein targeted for treatment inserted into the Pg OMV, and said vector being useful for treating injuries or disease in a mammalian subject, wherein said subject carries a deficiency in a gene encoding said protein or an overexpression thereof.

Yet another aspect of the present disclosure includes a method of treating a cancer in a subject comprising obtaining or having obtained an outer membrane vesicle (OMV) from Porphyromonas gingivalis (Pg), wherein the cancer type is modulated by special AT-rich binding protein-2 (SATB2) dysregulation and administering Pg OMV to patient.

### BRIEF DESCRIPTION OF THE DRAWINGS

The present disclosure relates to a method of gene targeting utilizing outer membrane vesicles (OMVs). More specifically, the present disclosure relates to a utilizing bacterially derived OMVs to modulate expression of specific mammalian genes.

The foregoing and other features and advantages of the present disclosure will become apparent to one skilled in the art to which the present invention relates upon consideration of the following description of the invention with reference to the accompanying drawings, wherein like reference numerals refer to like parts unless described otherwise throughout the drawings and in which:

FIG. 1 is an illustration of a table showing results of a metagenomic analysis with DNase treated Porphyromonas gingivalis (Pg) outer membrane vesicles (OMVs);

FIG. 2A is a figure representing treatment in a mouse neural progenitor cell with Pg OMVs resulting in decreased Neuregulin 1 (NRG1), according to one embodiment of the present disclosure;

FIG. 2B is a chart representing fluorescence intensity Fluorescein-5-isothiocyanate (FITC) v. DAPI (4′,6-diamidino-2-phenylindole) of Neuregulin 1 (NRG1), according to one embodiment of the present disclosure;

FIG. 2C is a chart representing fluorescence intensity Fluorescein-5-isothiocyanate (FITC) v. DAPI (4′,6-diamidino-2-phenylindole) of Beta-actin, according to one embodiment of the present disclosure;

FIG. 3 is a figure representing brain and placenta sizes of offspring exposed to Pg OMVs in utero, according to one embodiment of the present disclosure;

FIG. 4A is a figure representing NRG1 protein levels in a brain of offspring exposed to Pg OMVs in utero, according to one embodiment of the present disclosure;

FIG. 4B is a chart representing florescence intensity of Cy5 v. DAPI (4′,6-diamidino-2-phenylindole) of Neuregulin 1 (NRG1), according to one embodiment of the present disclosure;

FIG. 4C is a chart representing relative gene expression NRG1 mRNA levels, according to one embodiment of the present disclosure;

FIG. 5 is a figure representing NRG1 messenger ribonucleic acid protein (mRNA) levels and cytokine expression levels in offspring exposed to Pg OMVs in utero, according to one embodiment of the present disclosure;

FIG. 6A is a figure representing altered neuronal development in the cortex of mice offspring exposed to Pg OMVs in utero, according to one embodiment of the present disclosure;

FIG. 6B shows charts representing an indicated fluorescence intensity v. DAPI (4′,6-diamidino-2-phenylindole) of Cux1, SatB2, and Ctip2 genes according to one embodiment of the present disclosure;

FIG. 7 is a figure representing Pg OMVs internalized by neuronal cells, according to one embodiment of the present disclosure;

FIG. 8A is a chart showing Pg OMVs not eliciting an Interleukin-1 beta response, according to one embodiment of the present disclosure;

FIG. 8B is a chart showing Pg OMVs not eliciting an Interleukin-6 beta response, according to one embodiment of the present disclosure;

FIG. 8C is a chart showing Pg OMVs not eliciting an Interferon gamma response, according to one embodiment of the present disclosure;

FIG. 8D is a chart showing Pg OMVs not eliciting a Tumor Necrosis Factor alpha response, according to one embodiment of the present disclosure;

FIG. 8E is a chart showing Pg OMVs not eliciting a Myeloid differentiation primary response 88 response, according to one embodiment of the present disclosure;

FIG. 8F is a chart showing Pg OMVs not eliciting a NF-kappaB inhibitor 1 response, according to one embodiment of the present disclosure;

FIG. 8G is a chart showing Pg OMVs not eliciting a NF-kappaB inhibitor 2 response, according to one embodiment of the present disclosure;

FIG. 8H is a chart showing Pg OMVs not eliciting a NLRP3 inflammasome response, according to one embodiment of the present disclosure;

FIG. 9 is a chart showing Pg OMVs not eliciting expression of Hypoxia-inducible factor 1-alpha, according to one embodiment of the present disclosure;

FIG. 10A is a chart showing Pg OMVs not eliciting a tumor necrosis factor alpha gene response in placentas of dams exposed to Pg OMVs, according to one embodiment of the present disclosure;

FIG. 10B is a chart showing Pg OMVs not eliciting an Interleukin-1 beta gene response in placentas of dams exposed to Pg OMVs, according to one embodiment of the present disclosure;

FIG. 10C is a chart showing Pg OMVs not eliciting an Interferon alpha gene response in placentas of dams exposed to Pg OMVs, according to one embodiment of the present disclosure;

FIG. 10D is a chart showing Pg OMVs not eliciting an Interleukin-6gene response in placentas of dams exposed to Pg OMVs, according to one embodiment of the present disclosure;

FIG. 11A is a chart showing Pg OMVs not eliciting a Splicing factor 1 gene response associated with preterm birth and preeclampsia, according to one embodiment of the present disclosure;

FIG. 11B is a chart showing Pg OMVs not eliciting a Vascular endothelial growth factor A gene response associated with preterm birth and preeclampsia, according to one embodiment of the present disclosure;

FIG. 11C is a chart showing Pg OMVs not eliciting a Placental growth factor gene response associated with preterm birth and preeclampsia, according to one embodiment of the present disclosure;

FIG. 12 is a chart showing a cytokine array of dam livers exposed to Pg OMVs, according to one embodiment of the present disclosure; and

FIG. 13 shows charts showing astrocytes that are resistant to Pg OMVs, according to one embodiment of the present disclosure.

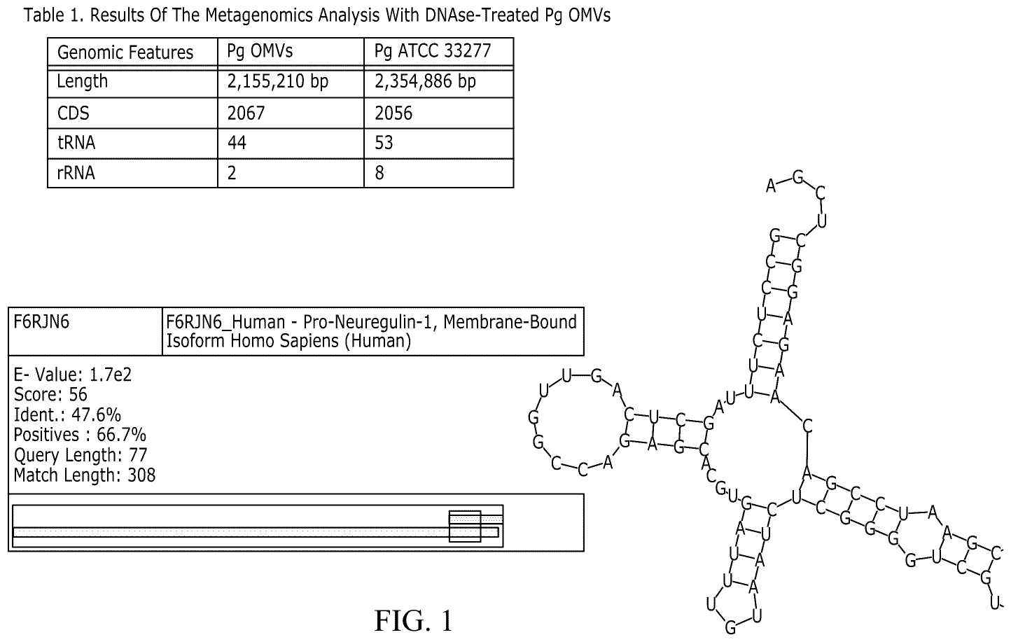

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

Using shotgun metagenomics, sequenced deoxyribonucleic acid (DNA) is extracted from naturally produced Porphyromonas gingivalis (Pg) outer membrane vesicles (OMVs) and revealed that specific genes were absent, which suggests that DNA is specifically packaged within Pg OMVs. In addition, genetic material that targets a specific mammalian host gene was identified, and in vitro and in vivo studies revealed that the specific mammalian host gene, and its downstream effectors develop negative outcomes when treated with Pg OMVs. Herein, a novel pathogenic mechanism by which Pg OMVs influences host biology is disclosed.

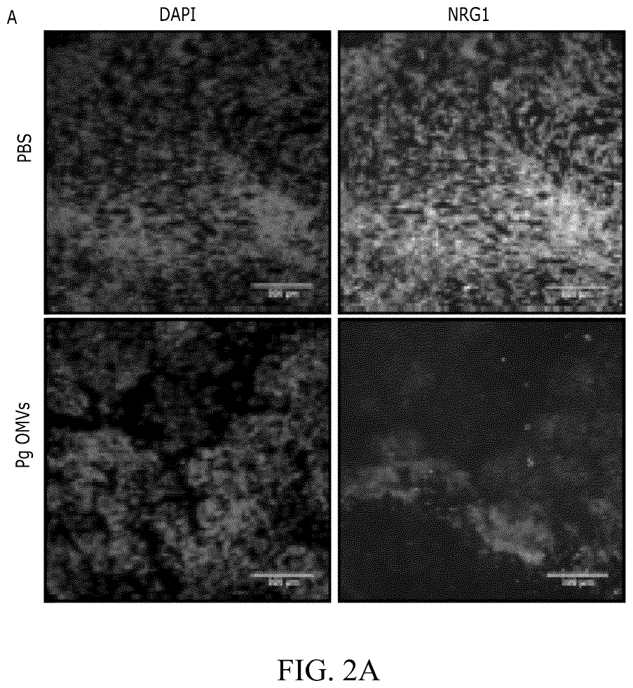

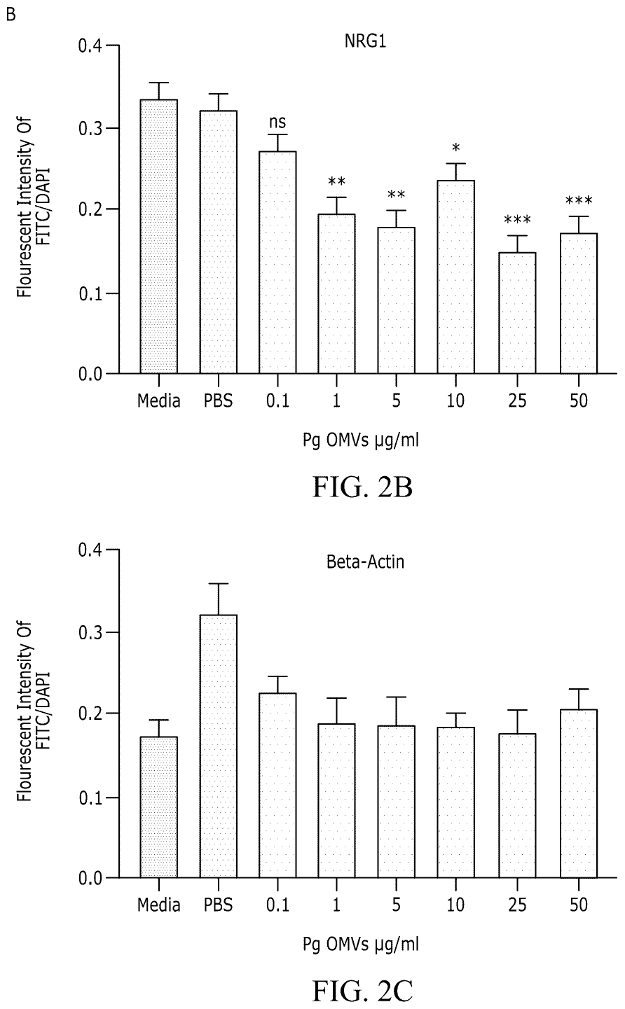

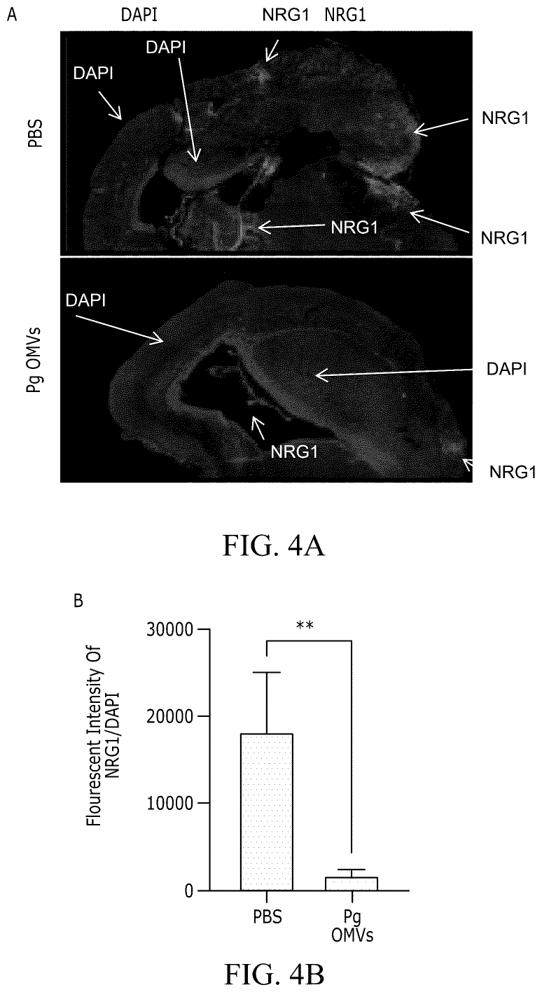

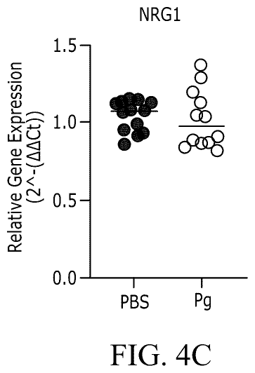

Herein a method by which bacterial OMVs modulate the expression of the mammalian gene neuregulin-1 (NRG1), a key growth factor in neuronal development and cell growth, is disclosed. Bacteria naturally produce OMVs that contain proteins, lipids, nucleic acids, and cytosolic compounds. Porphyromonas gingivalis (Pg) and closely related oral pathogens induce periodontitis and produce OMVs. Metagenomic sequencing revealed that Pg OMVs contain a transfer ribonucleic acid (tRNA) that is common among members of the phylum Bacteroidetes, other oral pathogens, and has complementarity to mammalian NRG1 messenger ribonucleic acid (mRNA). The complementation leads to a direct interaction between the Pg tRNA and NRG1 mRNA which results in a reduction of NRG1 translation. As illustrated in FIGS. 2A-2B, 4A, both in vitro and in vivo, there is a reduction in NRG1 when cultured cells and mice are exposed to Pg OMVs, relative to unexposed mice.

In FIG. 2A, representative images of mouse neural progenitor cells (NPC) are illustrated. Under DAPI and NRG1, there are PBS and Pg OMV treated NPCs. DAPI binds strongly to adenine-thymine-rich regions in DNA, illustrating the concentration of DNA present in NPCs treated with PBS and Pg OMVs, while a fluorescent dye specific to NRG1 illustrates the concentration of NRG1 present NPCs treated with PBS and Pg OMVs.

FIG. 2B is a chart representing fluorescence intensity of Fluorescein-5-isothiocyanate (FITC) v. DAPI of NRG1 indicating a concentration of NRG1 genes in media, PBS, and various concentrations of Pg OMVs. As illustrated in FIGS. 2A and 2B, exposure of NPCs to Pg OMVs (at a concentration of 10 μg/ml Pg OMV proteins) results in reduced NRG1 expression.

FIG. 2C is a chart representing fluorescence intensity FITC v. DAPI indicating a concentration of Beta-actin protein in media, PBS, and various concentrations of Pg OMVs. As can be seen from FIG. 2C, concentrations of beta-actin protein are reduced in NPCs exposed to Pg OMVs but not significant in comparison to NRg1 which was significantly reduced (see FIG. 2B).

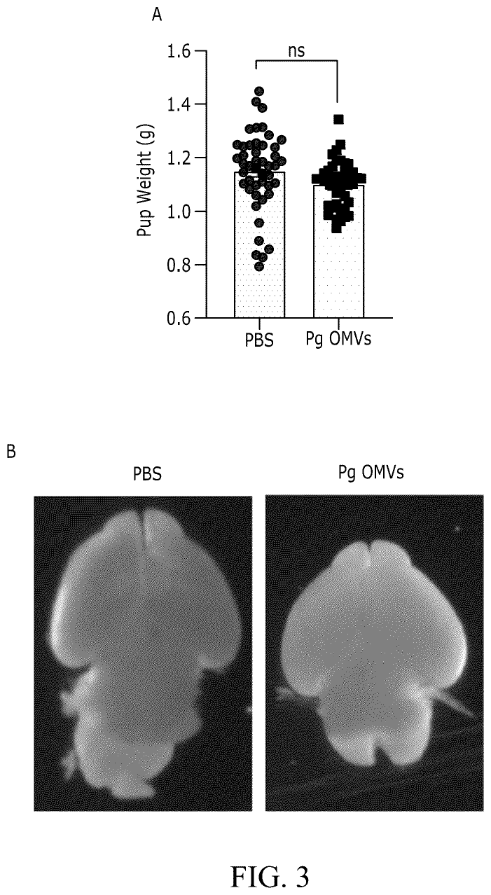

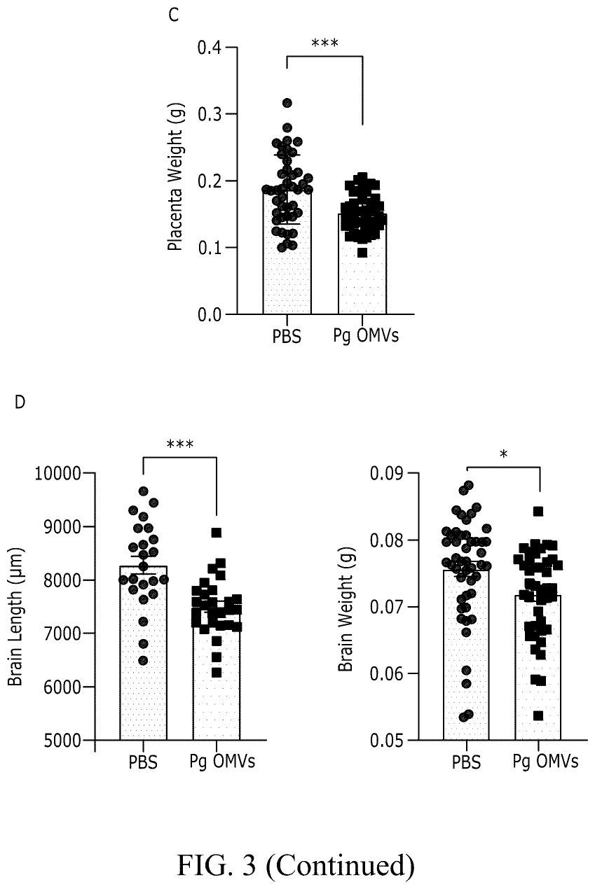

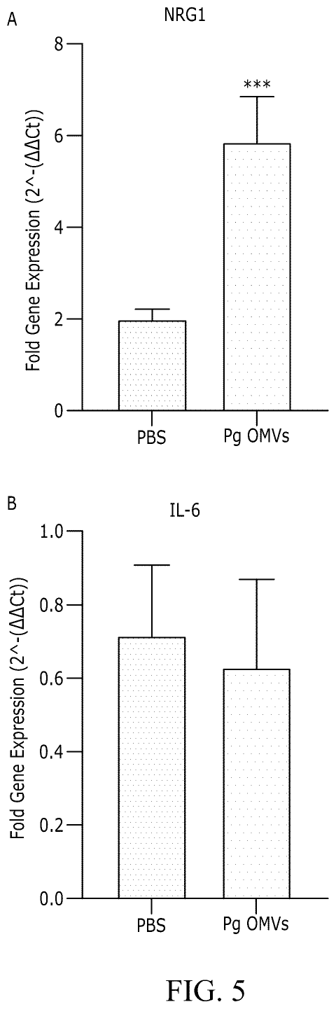

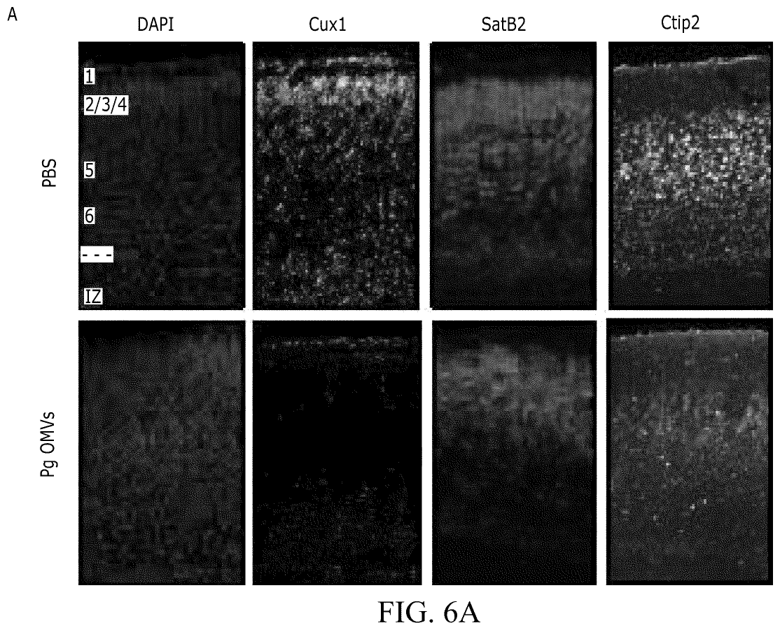

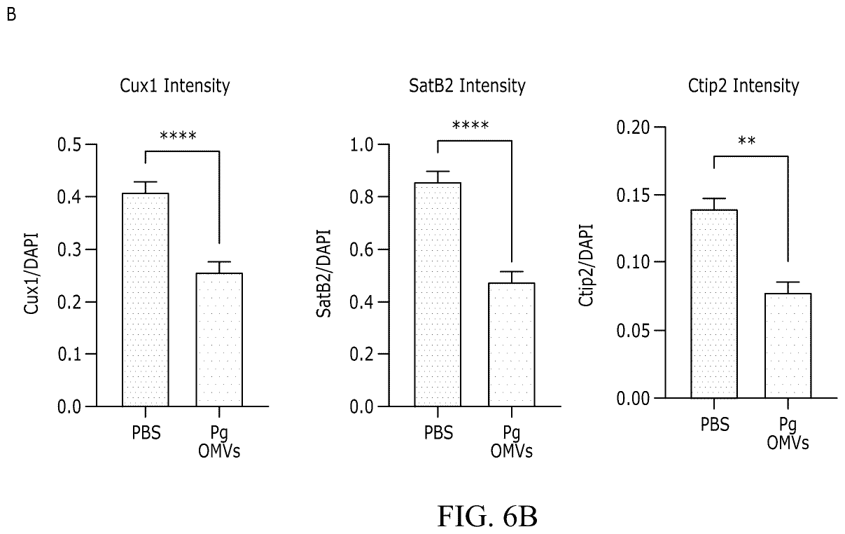

As illustrated in FIGS. 3-6B, genetic material found in naturally occurring Pg OMVs modulates gene expression of NRG1 in mammalian cells, as well as reducing Cux1 (a tumor suppressing gene), SatB2 (encodes for a protein that is involved in the development of the brain and structures in the head and face and is a therapeutic target for cancer), and Ctip2 (a transcription factor and involved in neuronal cell differentiation) intensities.

As illustrated in FIGS. 4A-4B, NRG1 expression in pups of mice exposed to Pg OMVs during pregnancy (Pg OMV pups) is reduced. In FIG. 4A, representative images of cortical regions of Pg OMV pup brains are illustrated. Under DAPI and NRG1, there are PBS and Pg OMV treated Pg OMV pup brains. DAPI illustrates the concentration of DNA present in cortical regions of Pg OMV pup brains treated with PBS and Pg OMVs, while a fluorescent antibody specific to NRG1 illustrates the concentration of NRG1 proteins present in cortical regions of Pg OMV pup brains of mothers exposed to PBS and Pg OMVs. FIG. 4B is a chart representing fluorescence intensity ration of Cy5 (far-red-fluorescent label for protein and nucleic acid conjugates) v. DAPI of NRG1 indicating a concentration of NRG1 genes in cortical regions of Pg OMV pup brains treated with PBS and Pg OMVs, respectively. As illustrated in FIGS. 4A and 4B, exposure of mothers treated with Pg OMVs (at a concentration of 50 μg/ml) results in reduced NRG1 expression in cortical regions of Pg OMV pup brains.

The Pg OMVs contain proteins and lipids that allow said Pg OMVs to cross mammalian cell membranes, as well as placental and blood-brain barriers. Additionally, Pg OMVs contain genetic material that produces protein translation and tRNA synthesis machinery, and tRNAs. Unlike infused recombinant NRG1 treatments, OMV modulated NRG1 expression affects intracellular NRG1 mediated functions in addition to autocrine and paracrine signaling to support cell development, differentiation, and growth.

The Pg OMVs are usable to modify the nucleic acid content, virulence factors of Pg, commensal or probiotic bacteria that modulate NRG1 expression via the OMVs or components thereof for a treatment that: 1) supports neurodevelopment in offspring of mothers; 2) reduces risk of pre-term and low birth weights for mothers; 3) ameliorates gestational metabolic disease in mothers; 4) ameliorates liver, kidney, and cardiac diseases in small animals, and/or 5) reduces the risk of pre-term birth and low-birth weights in small animals. Treatments 1 through 5 are for mothers with and without periodontal disease. The Pg OMVs are usable for a treatment that: 1) induces expression of NRG1 for various diseases; 2) inhibits expression of NRG1 for various diseases; and/or 3) alters functionality of NRG1 for various diseases.

Metagenomic sequencing was performed on DNase treated OMVs from Pg ATCC 33277. Pg OMVs were incubated with mouse neural progenitor cells, neurons, astrocytes and oligodendrocytes. Pg OMVs were incubated with human neural progenitor cells, neurons, astrocytes, and oligodendrocytes. Pg OMVs were incubated with 3D neurospheres composed of human neural progenitor cells, neurons, astrocytes and oligodendrocytes. Pg OMVs were tail vein injected into pregnant C57/Bl6 mice and collected pups at gestational age 18 (GA 18). Pg tRNA was confirmed to be in the amniotic fluid of mice exposed to Pg OMVs. Brains from GA 18 pups were sectioned and stained for markers of neuronal migration, development and NRG1. Brains from GA18 pups were dissected into front, middle, and hind regions and their nucleic acids and proteins were extracted to quantitate gene and protein expression of NRG1. Heart, spleen, liver and kidneys, placentas were collected from pregnant dams at GA 18.

Metagenomics revealed that a tRNA found in Pg OMVs has the potential to target NRG1 and alter translation. As illustrated in FIGS. 3, 4A-4C, 6A-6B, exposure to Pg OMVs in utero results in smaller brains and placentas in mouse offspring. NRG1 protein levels are decreased in neural progenitor cells in vitro and brains in vivo after exposure to Pg OMVs. In FIG. 4C, NRG1 mRNA levels are not increased in brains after Pg OMV treatment, which suggests translation of NRG1 mRNA is being affected. NRG1-mediated neuronal migration is altered in the brains of mouse offspring inoculated with Pg OMVs.

Advantageously, Pg OMVs have the ability to cross the placenta and blood-brain barriers in addition to mammalian cell membranes.

Unlike infused NRG1 treatments, Pg OMVs have the ability to modulate the expression of NRG1 intracellularly, which affects intracellular NRG1 mediated functions in addition to autocrine and paracrine signaling that support cell development, differentiation, and growth. Additionally, probiotic bacteria and/or Pg OMVs modify the expression of NRG1 and the expression of genes other than NRG1. Further, probiotic bacteria modify gene expression more locally where OMVs do not need to cross the placenta or blood-brain barrier. Additionally, Pg OMV's can be combined with other therapies to modify or improve treatment outcomes.

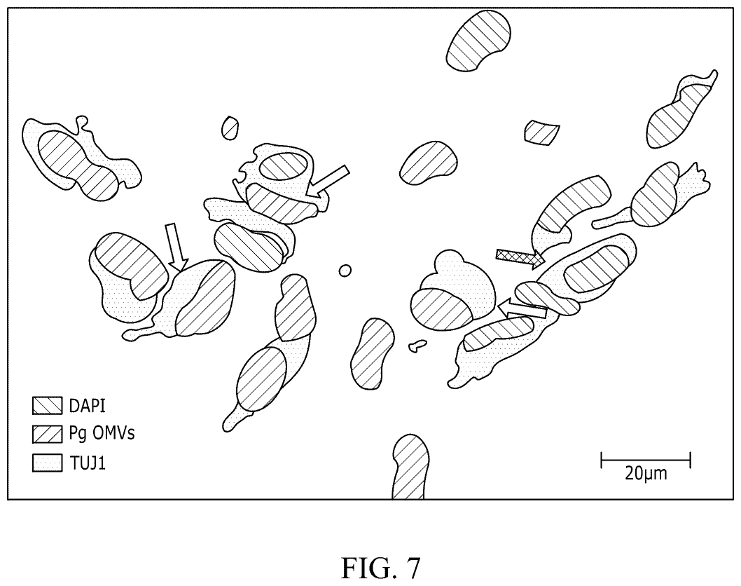

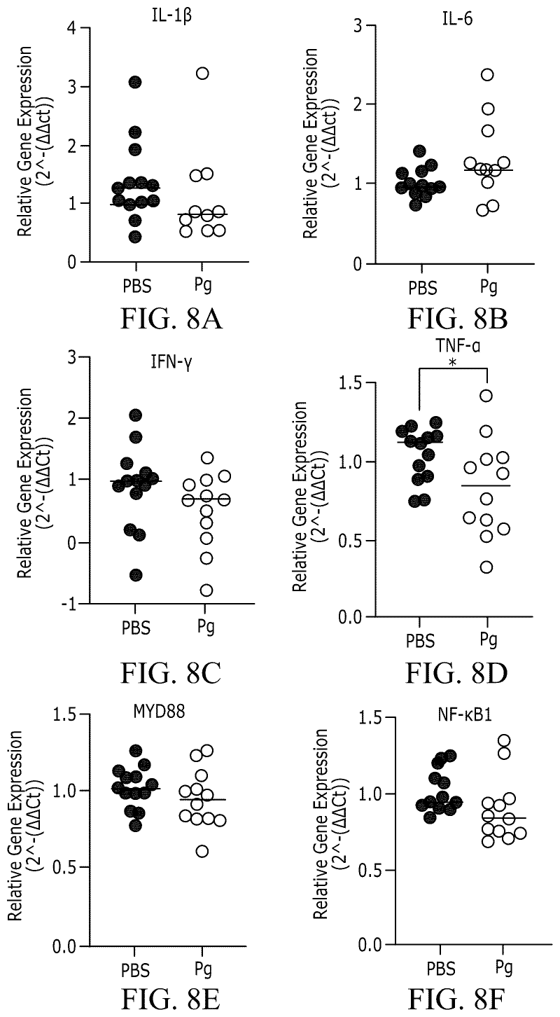

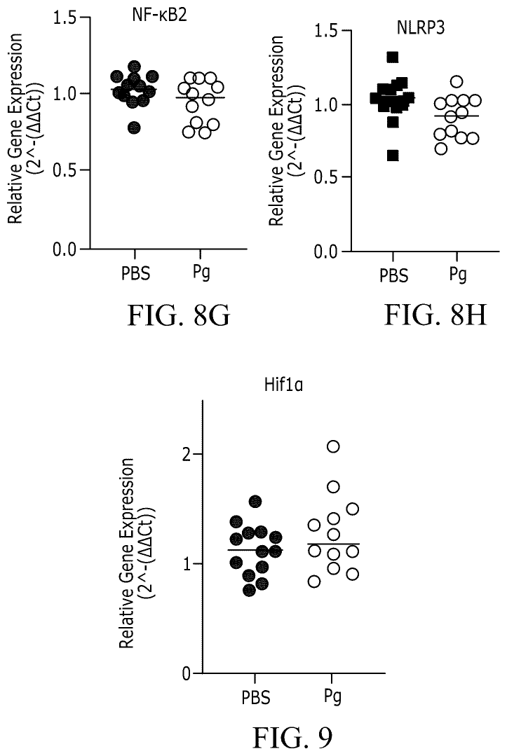

As illustrated in FIG. 8, Pg OMVs are internalized by neuronal cells. In FIG. 7, representative images of neuronal cells treated with Pg OMVs are illustrated. The cells illustrated are mouse neuronal cells that were grown on coverslips and treated with 10 μg/ml DiO labeled Pg OMVs for 1 hour. The mouse neuronal cells were washed, fixed, and probed with α-TUJ1 followed by staining with DAPI and a secondary antibody conjugated to alexafluor 546. Coverslips were imaged at 63× on an Apotome 3 fluorescent microscope. OMV's were presented on the cell surface, as indicated by arrows. Pg OMVs are illustrated as present on the cell surface 702 as well as internalized and localized with the nucleus.

Pg OMVs are a vector for gene delivery. In one example embodiment, Pg OMVs function as a gene in situ or in vivo gene delivery system, such as to hepatic and/or neuronal cells, advantageously being up taken by neuronal cells (see FIG. 7) Therapeutic genes, such as RNA (ribonucleic acid), mRNA (messenger RNA), siRNA (silencing RNA), tRNA (transfer RNA), deoxyribonucleic acid (DNA), and/or cDNA (copy DNA), are insertable into the Pg OMVs, wherein the Pg OMVs can deliver the therapeutic genes across the placenta and/or the blood brain barrier. Further, DNA and RNA vaccine candidates are insertable into the Pg OMV for delivery of single or double stranded ribonucleic acid (e.g., RNA or DNA).

As illustrated in FIGS. 8A-8H, Pg OMVs do not elicit an immune response in mouse brains, and actually tamp down immune responses. As illustrated in FIGS. 8A-8H, relative gene expression in mouse brains exposed to either PBS or Pg OMVs for immune proteins Interleukin-1 beta, Interleukin-6 beta, Interferon gamma, Interferon alpha, Myeloid differentiation primary response 88, NF-kappaB inhibitor 1, NF-kappaB inhibitor 2, and NLRP3 inflammasome was measured, and the immune response was lower in the presence of Pg OMVs than in PBS. As such, Pg OMVs function as treatment for diseases that cause harmful or excessive immune responses, as Pg OMVs reduce or tamp down immune responses,

As illustrated in FIG. 9, Pg OMVs do not induce expression of hypoxia-inducible factor 1-alpha, a gene that responds to hypoxia. In FIG. 9, relative gene expression in mouse brains exposed to either PBS or Pg OMVs for hypoxia-inducible factor 1-alpha in utero remained unchanged.

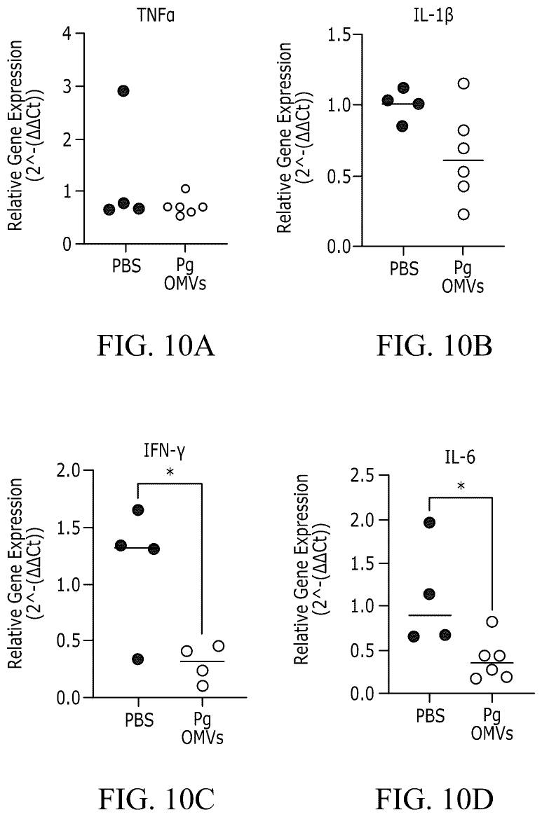

As illustrated in FIGS. 10A-10D, Pg OMVs do not elicit an immune response in placentas of dams, and actually tamp down immune responses. As illustrated in 10A-10D, relative gene expression in mouse placentas exposed to either PBS or Pg OMVs for immune proteins tumor necrosis factor alpha, Interleukin-1 beta, Interleukin-6 beta, and Interferon alpha was measured, and the immune response was lower in the presence of Pg OMVs than in PBS. As such, Pg OMVs also function as treatment for diseases that cause harmful or excessive immune responses in fetuses, as Pg OMVs reduce or tamp down immune responses.

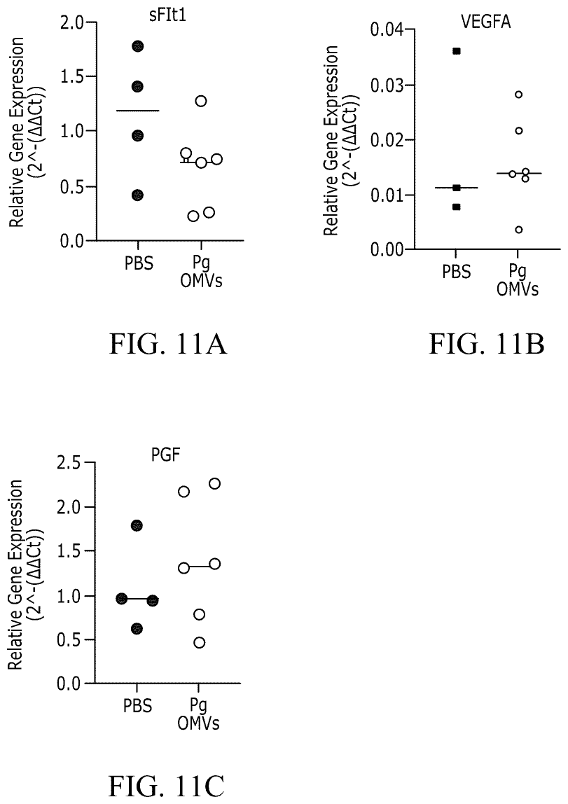

As illustrated in FIGS. 11A-11C, Pg OMVs do not elicit an decrease in growth factors associate with preterm birth or preeclampsia. As illustrated in 11A-11C, relative gene expression in mouse placentas exposed to either PBS or Pg OMVs for growth factors, Splicing factor 1, Vascular endothelial growth factor A, and Placental growth factor the growth factor response was essentially the same in the presence of Pg OMVs and PBS.

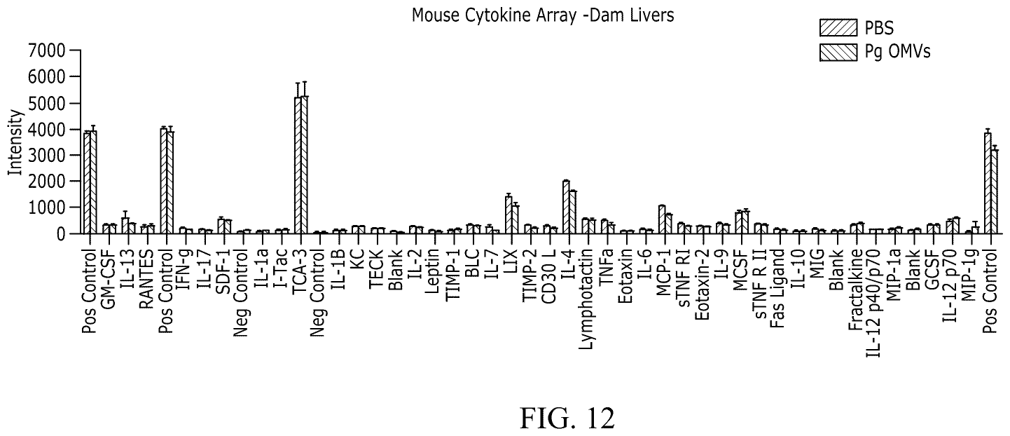

As illustrated in FIG. 12, a cytokine array of dam livers exposed to either PBS or Pg OMVs, the cytokine array measured granulocyte macrophage colony-stimulating factor (GM-CSF), interleukin-13 (IL-13), Regulated upon Activation, Normal T Cell Expressed and Presumably Secreted (RANTES), interferon gamma (IFN-g), Interleukin-17 (IL-17), Stromal cell-derived factor-1 (SDF-1), Interleukin-1 alpha (IL-1 alpha), interferon-inducible T cell alpha chemoattractant (I-TAC), TCA3 (a pro-inflammatory cytokine), Interleukin-1 beta (IL-1 beta), keratinocyte-derived cytokine (KC), Thymus-Expressed Chemokine (TECK), Interleukin-2 (IL-2), Leptin, TIMP-1 (triggers a proinflammatory phenotype in human monocytes), B lymphocyte chemoattractant (BLC), Interleukin-7 (IL-7), LIX (proinflammatory cytokine), TIMP-2 (triggers a proinflammatory phenotype in human monocytes), CD30 (membrane-bound cytokine), Interleukin-4 (IL-4), lymphotactin, Tumor necrosis factor alpha (TNF-α), Eotaxin, Interleukin-6 (IL-6), Monocyte chemoattractant protein-1 (MCP-1), soluble TNF-α receptors (sTNFRr1), Eotaxin-2, Interleukin-9 (IL-9), Macrophage colony-stimulating factor (M-CSF), soluble TNF-α receptors (sTNFR II), Fas ligand, Interleukin-10 (IL-10), Monokine induced by gamma (MIG), Fractalkine, Interleukin-12 (IL-12 p40/p70), Macrophage inflammatory protein (MIP-la), granulocyte colony-stimulating factor (G-CSF), Interleukin-12 (IL-12 p70), Macrophage Inflammatory Protein-1 gamma (MIP-1g), and positive and negative controls. FIG. 12 further illustrates that Pg OMVs tamp down immune responses, that is causes immune responses to be less than they would be in normal or control circumstances.

Pg OMV reduce neuroinflammation in general. In traumatic brain injury (TBI) patients, initial inflammation is potentially helpful to TBI patients, so long as it is not prolonged. Administration of Pg OMV's will decrease the inflammation in a TBI patient, when indicated by a physician.

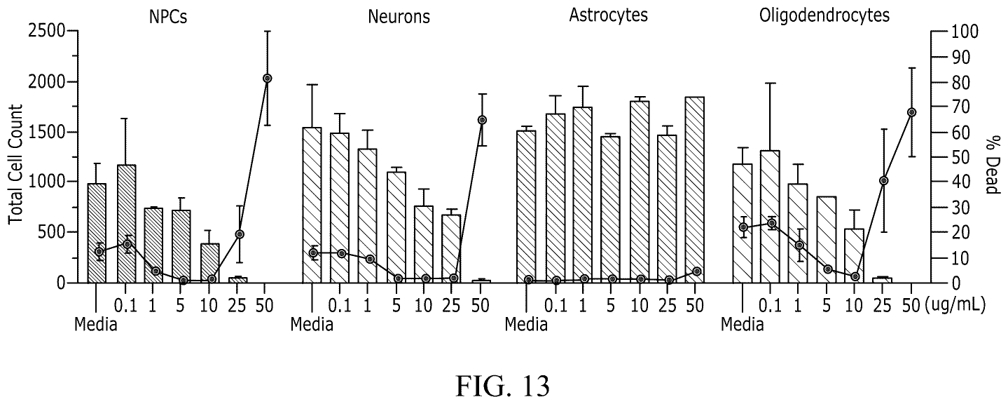

As illustrated in FIG. 13, human neural progenitor cells (NPCs) having been differentiated into different neural cell types and treated with Pg OMVs at the indicated concentrations (e.g., 0.1, 1, 5,10, 25, 50 μg/mL) every other day for a total of 7 days. Wells were imaged and quantified with a micro confocal imaging system. One such example imaging system is the ImageXpress® Micro Confocal High-Content Imaging System from Molecular Devices LLC. Total cell counts (DAPI) and percent dead cells (LIVE or Dye viability stain) were plotted. FIG. 13 illustrates that NPCs, neurons, and oligodendrocytes were susceptible to Pg OMVs whereas astrocytes were resistant. Thus, Pg OMVs are administrable to promote astrocytes, and/or wherein neurons, NPCs and/or oligodendrocytes are over replicating (e.g., such as in certain types of brain cancer).

In the foregoing specification, specific embodiments have been described. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected or in contact, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter. All documents referenced herein are incorporated by reference in their entireties for all purposes.

What is claimed is:

1. A method of altering NRG1 expression in a subject in need thereof, the method comprising:
isolating an outer membrane vesicle (OMV) from Porphyromonas gingivalis (Pg), the Pg OMV containing transfer ribonucleic acid (tRNA) complementary to NRG1 messenger ribonucleic acid (mRNA); and
administering to the subject the Pg OMV.

2. The method of claim 1, further comprising a method of treating a neurodevelopment issues in subject in utero by administering to a mother of the subject the Pg OMV.

3. A Pg OMV isolated by the method of claim 1.

4. A method of treating a cancer in a subject comprising administering to the subject a Pg OMVs generated by the method of claim 1, or administering the Pg OMV of claim 3.

5. A method of treating metastatic cancer in a subject comprising administering to the subject the Pg OMVs generated by the method of claim 1, or administering the Pg OMV of claim 3.

6. A method of treating autoimmune disease, in a subject comprising administering to the subject the Pg OMVs generated by the method of claim 1, or administering the Pg OMV of claim 3.

7. A method of treating hepatic autoimmune disease, in a subject comprising administering to the subject the Pg OMVs generated by the method of claim 1, or administering the Pg OMV of claim 3.

8. A method of reducing oligodendrocytes, human neural progenitor cells and neurons while promoting astrocytes in a subject comprising administering to the subject a Pg OMVs generated by the method of claim 1, or administering the Pg OMV of claim 3.

9. A method of treating traumatic brain injury induced inflammation in a subject comprising administering to the subject a Pg OMVs generated by the method of claim 1, or administering the Pg OMV of claim 3.

10. Gene therapy vector for expressing an exogenous nucleic acid sequence comprising:
an outer membrane vesicle (OMV) from Porphyromonas gingivalis (Pg);
a nucleic acid sequence encoding for a protein targeted for treatment inserted into the Pg OMV; and
said vector being useful for treating injuries or disease in a mammalian subject, wherein said subject carries a deficiency in a gene encoding said protein or an overexpression thereof.

11. The gene therapy vector of claim 10, wherein the nucleic acid sequence is one of ribonucleic acid (RNA), messenger RNA, silencing RNA, tRNA (transfer RNA), deoxyribonucleic acid (DNA), and cDNA (copy DNA)

12. The gene therapy vector of claim 10, wherein the nucleic acid sequence encodes for a neuronal protein, further wherein the Pg OMV is internalized by neuronal cells.

13. The gene therapy vector of claim 10, wherein the nucleic acid sequence encodes for a protein utilized in placental or fetal development, further wherein the Pg OMV is enters a placenta of a fetus through administration to a mother of the fetus.

14. The gene therapy vector of claim 11, wherein the nucleic acid sequence encodes for a protein utilized in placental or fetal development, further wherein the Pg OMV is enters a placenta of a fetus through administration to a mother of the fetus.

15. A method of treating a cancer in a subject comprising administering to the subject Pg OMV of claim 10.

16. A method of treating a cancer in a subject comprising administering to the subject Pg OMV of claim 11.

17. A method of treating autoimmune disease in a subject comprising administering the Pg OMV of claim 10.

18. A method of treating a cancer in a subject comprising:
(a) obtaining or having obtained an outer membrane vesicle (OMV) from Porphyromonas gingivalis (Pg), wherein the cancer type is modulated by special AT-rich binding protein-2 (SATB2) dysregulation; and
(b) administering Pg OMV to patient.

19. A Pg OMV obtained by the method of claim 17.

20. The method of claim 18, wherein the Pg OMVs are characterized utilizing nanoparticle-tracking analysis (NTA) and transmission electron microscopy (TEM).
